(19) United States
(12) Patent Application Publication
(10) Pub. No.: US 2023/0256271 A1
(43) Pub. Date: Aug. 17, 2023

(54) SYMMETRICAL FLOW RESPIRATOR

(71) Applicant: XCMR Inc., Narberth, PA (US)

(72) Inventor: Richard A. Rasansky, Narberth, PA (US)

(21) Appl. No.: 18/169,096

(22) Filed: Feb. 24, 2023

Related U.S. Application Data
(60) Provisional application No. 63/310,019, filed on Feb. 14, 2022.

(51) Int. Cl. A62B 18/10; A62B 18/02

(52) U.S. Cl. CPC A62B 18/10; A62B 18/025; A61M 16/20

(57) ABSTRACT

A protective respirator, including an inhalation reactor chamber that treats and provides intake air to a user and an exhaust reactor chamber that treats exhaust air exhaled by the user. The respirator uses ultraviolet C radiation to deactivate potentially harmful particles without exposing the user to the radiation. In embodiments, the respirator includes a number of features that increase efficiency and prolong battery life. For instance, the protective respirator may use approximately half the power by alternating between the inhalation and exhalation reactor chambers. Additionally, the respirator may adjust the intensity of the UVC radiation based on the intensity of the user's respiration. Additionally, the reactor chamber cavities may be lined with a reflective material that exponentially increases the irradiance inside each chamber cavity. Additionally, each reactor chamber cavity may include fluid-permeable photon barriers that reflect UVC photons back into the reactor chamber cavity while allowing airflow to pass.

### CROSS-REFERENCE TO RELATED APPLICATIONS

This application claims priority to U.S. Prov. Pat. Appl. No. 63/310,019, filed Feb. 14, 2022, which is hereby incorporated by reference in its entirely.

### FEDERAL FUNDING

None

### BACKGROUND

From soldiers wearing Mission Oriented Protective Posture (MOPP) suits to civilians wearing masks to protect themselves from COVID-19, many individuals have a desire to protect themselves from inhaling pathogens and other potentially harmful particles and prevent themselves from spreading those particles.

Ultraviolet (UV) radiation having a wavelength between about 200 nanometers (nm) and 320 nm causes photochemical damage to critical biomolecules like deoxyribonucleic acid (DNA) and ribonucleic acid (RNA). Therefore, UV radiation in the 200-320 nm range, which is sometimes referred to as “germicidal UV,” can be used to disinfect surfaces and volumes of air. UV radiation having a wavelength less than about 240 nm also damages proteins, which can also lead to microbial and viral inactivation.

UV radiation, however, presents a number of technical challenges that have thus far prevented the development of practical, portable respirators that utilize UV radiation to deactivate particles. For one thing, radiation having a wavelength greater than 230 nm can be harmful to human tissue. Additionally, because UV radiation sources are energy inefficient, they generate excess heat and make it challenging to develop a portable device that can provide protection for long time periods using a battery.

### SUMMARY

In order to overcome those and other drawbacks of the prior art, a protective respirator is disclosed. In embodiments, the protective respirator includes two reactor chambers, each embedded with a source of ultraviolet C (UVC) radiation, that deactivate microbes and/or other potentially harmful particles without exposing the user to the UVC radiation. An inhalation reactor chamber that treats and provides intake air to a user (e.g., via a facemask) and an exhaust reactor chamber treats exhaust air exhaled by the user.

In various embodiments, the protective respirator includes a number of features that increase the efficiency and prolong the battery life of the device. For instance, the protective respirator may use approximately half the power by alternating between activating and deactivating the inhalation and exhalation reactor chambers depending on whether the user is inhaling or exhalating. Additionally, the protective respirator may adjust the intensity of the UVC radiation based on the intensity of the user's respiration. Additionally, the reactor chamber cavities may be lined with a reflective material, such as a microporous expanded polytetrafluoroethylene (ePTFE) material, exponentially increasing the irradiance inside each chamber cavity. Additionally, each reactor chamber cavity may include two fluid-permeable photon barriers that reflect UVC photons back into the reactor chamber cavity while allowing airflow to pass into and out of the reactor chamber cavity.

### BRIEF DESCRIPTION OF THE DRAWINGS

Aspects of exemplary embodiments may be better understood with reference to the accompanying drawings. The components in the drawings are not necessarily to scale, emphasis instead being placed upon illustrating the principles of exemplary embodiments.

FIG. 1 is a drawing which illustrates a personal protective respirator according to exemplary embodiments.

FIG. 2 is a block diagram of the protective respirator of FIG. 1 according to exemplary embodiments.

FIG. 3A is the action spectrum of the SARS-CoV-2 virus.

FIG. 3B is the spectral distribution of a low pressure mercury (LPM) lamp.

FIG. 3C is the spectral distribution of UVC LEDs.

FIG. 4A is a schematic diagram of a reactor chamber cavity according to an exemplary embodiment where the UVC radiation source is an LPM lamp.

FIG. 4B is a schematic diagram of a reactor chamber cavity according to another exemplary embodiment where the UVC radiation source is an LPM lamp.

FIG. 4C is a diagram of a reactor chamber cavity according to another exemplary embodiment where the UVC radiation source is an array of UVC LEDs.

FIG. 5A is a diagram illustrating fluid-permeable photon barriers according to a first exemplary embodiment.

FIG. 5B is a front view of a fluid-permeable photon barrier according to a second exemplary embodiment.

FIG. 5C is a side view of the fluid-permeable photon barriers according to the embodiment of FIG. 5B.

FIG. 6 is a flowchart illustrating a UV emission process according to an exemplary embodiment.

FIG. 7A is a graph of an example respiration pattern.

FIG. 7B is an example timing diagram of a binary signal indicating the timing of the respiration pattern of FIG. 7A.

FIG. 7C is a timing diagram of pulses activating an inhalation reactor chamber and an exhalation reactor chamber in response to the respiration pattern of FIG. 7A or the binary signal of FIG. 7B.

FIG. 8A illustrates an example laboratory experiment to measure the intrinsic kinetics of the inactivation response of aerosolized particles.

FIG. 8B illustrates a computer simulation of the inactivation response during the experiment of FIG. 8A.

FIG. 9A is a block diagram of an analog ballast for a LPM lamp according to an exemplary embodiment.

FIG. 9B is a circuit diagram of the analog ballast of FIG. 9A.

FIG. 10A is a block diagram of a ballast for an LPM lamp having an integrated circuit-based lamp controller according to an exemplary embodiment.

FIG. 10B is a circuit diagram of the IC-based ballast of FIG. 10A.

FIG. 11 is a block diagram of an IC-based ballast according to another exemplary embodiment.

FIG. 12A is a block diagram illustrating circuit controllers adjusting the intensity of UVC LEDs according to another exemplary embodiment.

FIG. 12B is a circuit diagram illustrating the circuit controllers of FIG. 12A.

FIG. 13 is a diagram of a network environment according to exemplary embodiments.

### DETAILED DESCRIPTION

Reference to the drawings illustrating various views of exemplary embodiments is now made. In the drawings and the description of the drawings herein, certain terminology is used for convenience only and is not to be taken as limiting the embodiments of the present invention. Furthermore, in the drawings and the description below, like numerals indicate like elements throughout.

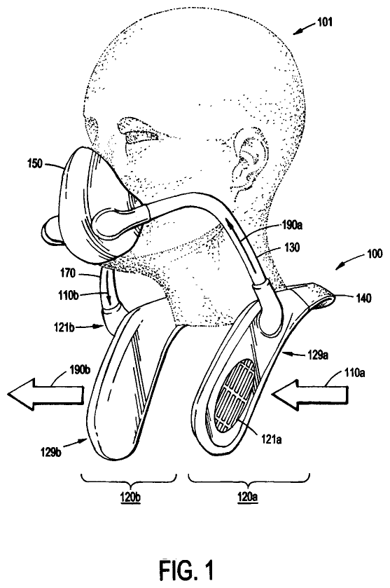

FIG. 1 illustrates a personal protective respirator 100 according to exemplary embodiments.

In the embodiments of FIG. 1, the protective respirator 100 is a “symmetrical flow” disinfection device that includes both an inhalation reactor chamber 120a and an exhalation reactor chamber 120b, which are each attached to a power/electronics pod 140. In those embodiments, the protective respirator 100 is realized as a neck collar that can be comfortably worn by a user 101 with the power/electronics pod 140 wrapping around the back of the neck of the user 101 and the reactor chambers 120 resting on the chest of the user 101.

The inhalation reactor chamber 120a and the exhalation reactor chamber 120b are each embedded with a source of ultraviolet C (UVC) radiation that deactivates microbes and/or other potentially harmful particles without exposing the user 101 to the UVC radiation. The inhalation reactor chamber 120a includes an intake air input port 121a for receiving untreated intake air 110a from the open atmosphere surround the user 101 and an intake air output port 129a, which is in flow communication with a facemask 150 (e.g., via an intake air tube 130), for outputting treated intake air 190a for inhalation by the user 101. Similarly, the exhalation reactor chamber 120b includes an exhaust air input port 121b that receives untreated exhaust air 110b from inside the facemask 150 (e.g., via an exhaust air tube 170) and an exhaust air output port 129b for outputting treated exhaust air 190b into the open atmosphere. The inhalation reactor chamber 120a and the exhalation reactor chamber 120b are collectively referred to herein as reactor chambers 120. The untreated intake air 110a and the untreated exhaust air 110b are collectively referred to herein as untreated air 110. The treated intake air 190a and the treated exhaust air 190b are collectively referred to herein as treated air 190.

In the embodiment of FIG. 1, the facemask 150 is a form-fitting mask configured to surround the nose and mouth of the user 101 and provide a sealed barrier between the nose and mouth of the user 101. In other embodiments, the facemask 150 may be a full face shield (for example, a helmet used by a pilot). In other embodiments, the facemask 150 may be configured to provide a barrier between the nose and mouth of the user 101 that is not necessarily sealed or air-tight.

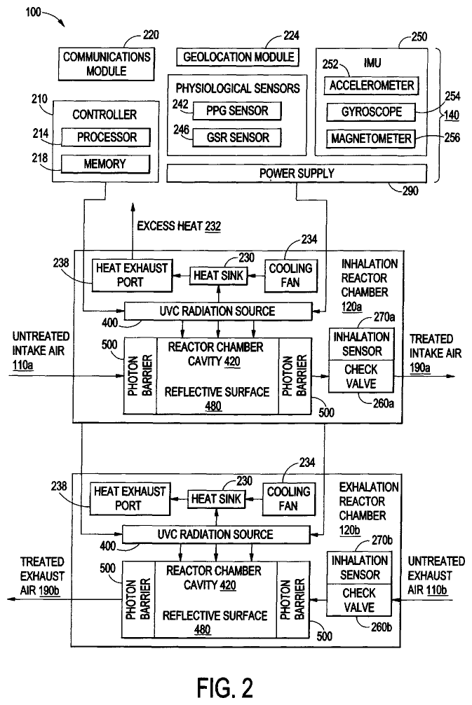

FIG. 2 is a block diagram of the protective respirator 100 according to exemplary embodiments.

The protective respirator 100 includes a controller 210, and a power supply 290. In the embodiment of FIG. 2, the power/electronics pod 140 also includes a communications module 220, a geolocation module 224, physiological sensors 240, and an inertial measurement unit 250.

The controller 210 may be any suitable computing device capable of performing the functions described herein. In the embodiment of FIG. 2, the controller 210 includes non-transitory computer readable storage media (memory 218) and a hardware computer processor 214. In other embodiments, the controller 210 may be, for example, a finite state machine. The power supply 290 may be any hardware device (for example, a rechargeable battery) that provides electrical power to the protective respirator 100.

The communications module 220 may be any hardware device enabling the protective respirator 100 to communicate with other electronic devices directly and/or via a network. For example, the communications module 220 may provide functionality for the protective respirator 100 to communicate with other protective respirators 100 and/or personal electronic devices (e.g., smartphones, activity monitors, fitness trackers, etc.) using direct, short range, wireless communication (e.g., Bluetooth). The geolocation module 224 may include any hardware device that determines or estimates the geographic position of the protective respirator 100 using, for example, satellite navigation, network identification, communication with location beacons, etc.

The physiological sensors 240 may include any hardware device that senses data indicative of the physiological condition of the user 101. The physiological sensors 240 may include a photoplethysmogram (PPG) sensor 242, which uses a light source and a photodetector at the surface of skin to measure the volumetric variations of blood circulation, and/or a galvanic skin response (GSR) sensor 246, which detects the changes in electrical (ionic) activity resulting from changes in sweat gland activity. Data from the PPG sensor 242 may be used, for example, to estimate the frequency, intensity, and amplitude of the respiration of the user 101.

The inertial measurement unit 250 may include any hardware device that measures and reports the motion and/or orientation of the protective respirator 200. The internal measurement unit 250 may include an accelerometer 252, a gyroscope 254, and/or a magnetometer 256. Motion of the protective respirator 100 may affect the data captured by the physiological sensors 240. Therefore, in some embodiments, the controller 210 may determine the motion of the protective respirator 100 based on data captured by the IMU 250 and use a digital signal processing algorithm (stored, for example, in the memory 118) to remove motion artifacts from the data captured by the physiological sensors 240.

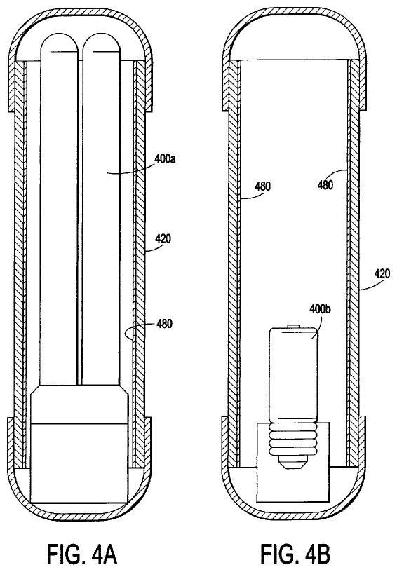

As described in detail below with reference to FIG. 4, each reactor chamber 120 includes a UVC radiation source 400 and a reactor chamber cavity 420 having a reflective surface 480. The untreated air 110 passes through reactor chamber cavity 420 where the untreated air 110 is exposed to UVC radiation emitted by the UVC radiation source 400, which deactivates microbes and/or other potentially harmful particles in the untreated air 110 to form the treated air 190. The UVC radiation source 400 may be, for example, a medium or low pressure mercury arc lamp, light emitting diodes (LEDs), laser diodes, an excimer lamp (e.g., an optically-filtered krypton-chloride (KrCl*) excimer lamp as described in U.S. patent application Ser. No. 17/644,041), an excimer laser, a microplasma lamp, etc.

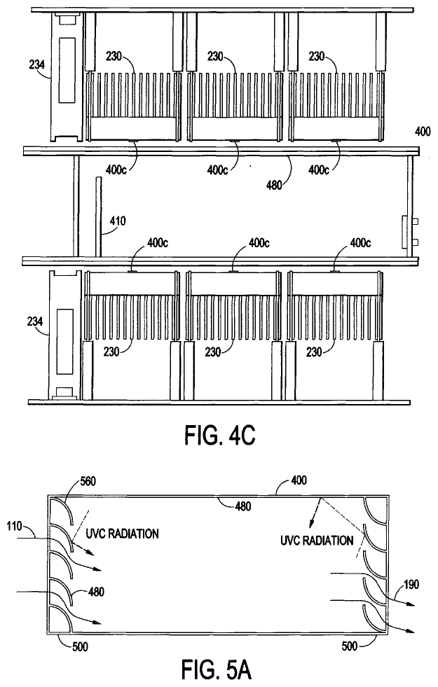

As described in detail below with reference to FIG. 5, each reactor chamber cavity 420 may include two fluid-permeable photon barriers 500 that reflect UVC photons back into the reactor chamber cavity 420 while allowing airflow to pass into and out of the reactor chamber cavity 420 with little obstruction.

To help regulate the temperature of the UVC radiation source, each reactor chamber 120 may include a heat sink 230 that transfers excess heat 232 generated by the UVC radiation source 400 to a fluid medium (e.g., a liquid coolant or air). In other embodiments, excess heat 232 may be transferred, for example, to a thermal paste, a conformal coating of copper with an electrical insulating layer of poly coating, a vapor chamber, etc. In some embodiments, the reactor chamber 120 may include a cooling fan 234 to dissipate the excess heat 232 out a heat exhaust port 232 and away from the reactor chamber 120. (In some embodiments, the exhalation reactor chamber 120b may vent excess heat 232 via the exhaust air output port 129b.)

As shown in FIG. 2, the inhalation reactor chamber 120a outputs the treated intake air 190a via a one-way check value 260a and the exhalation reactor chamber 120b receives the untreated exhaust air 110b via a one-way check value 260b. Each check valve 260a and 260b is configured to allow airflow in one direction and prevent reverse flow. For example, each of the check valves 260a and 260b may be configured to close unless opened by pressure in the direction of flow. For instance, the check valve 260a of the inhalation reactor chamber 120a is opened by the pressure created by the inhalation of the user 101 and closes when the pressure from the inhalation is no longer maintained. Similarly, the check valve 260b of the exhalation reactor chamber 120b is opened by the pressure created by the exhalation of the user 101 and closes when the pressure from the expiration is no longer maintained. Returning briefly to FIG. 1, the one-way check values 260a and 260b ensure a one-way airflow wherein treated intake air 190a flows into the facemask 150 after having been treated by the inhalation reactor chamber 120a and untreated exhaust air 110b flows out of the facemask 150 and into the open atmosphere after having been treated by the exhalation reactor chamber 120b.

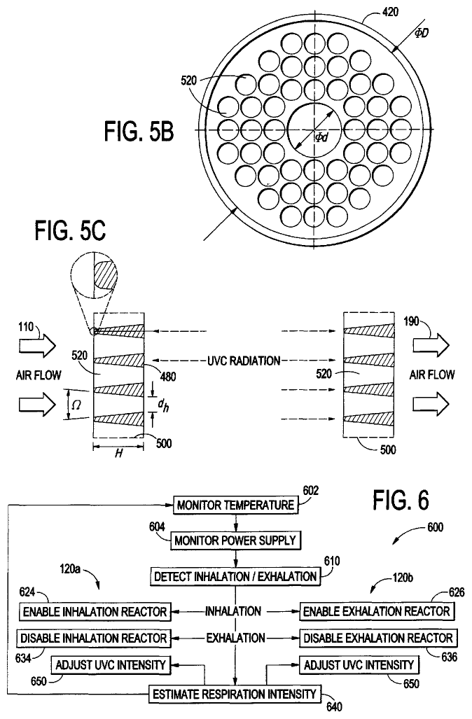

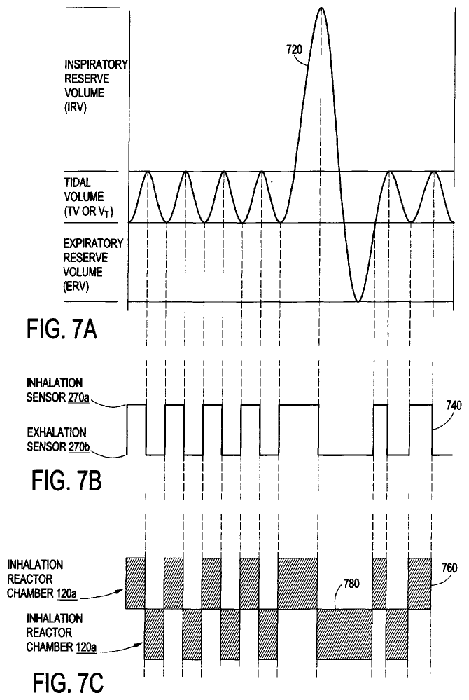

The inhalation reactor chamber 120a includes an inhalation sensor 270a and the exhalation reactor chamber 120b includes an exhalation sensor 270b. As described in detail below with reference to the flowchart of FIG. 6 and the timing diagrams of FIGS. 7A and 7B, each of the inhalation and exhalation sensors 270a and 270b capture and output information indicative of the timing of the respiration (i.e., inhalation or exhalation) of the user 101. In those embodiments, for example, each check valve 260a and 260b may include an integrated switch that opens or closes when the check valve 260a or 260b is opened. In other embodiments, the inhalation sensor 270a or exhalation sensor 270b may capture and output information indicative of the intensity of the inhalation or exhalation of the user 101. For example, the inhalation sensor 270a or exhalation sensor 270b may be a differential pressure sensor that measures the flow rate across the associated check valve 260a or 260b.

The protective respirator 100 is configured to deactivate at least one specific, potentially harmful particle in the untreated air 110 by emitting UVC radiation from the UVC radiation source 400. The specific particle may be, for example, a microorganism (e.g., a bacterium, a spore, a virus, a protozoon, and/or a fungus) or other aerosolized bio-threats (e.g., chemical agents). Because each particle has its own inherent action spectrum (i.e., absorption of UV radiation at different wavelengths) and each potential UVC radiation source has its own characteristic emission spectrum, the protective respirator 100 may include a UVC radiation source 400 configured to emit UVC radiation at a wavelength that is absorbed by the specific particle.

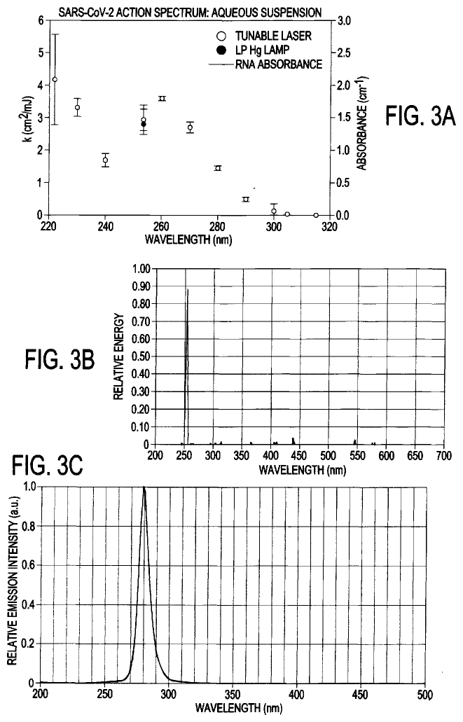

FIG. 3A is an action spectrum illustrating the ribonucleic acid (RNA) absorbance of the SARS-CoV-2 virus at wavelengths along the spectrum. FIG. 3B is a spectral distribution illustrating the relative emission intensity of a low pressure mercury (LPM) lamp at wavelengths along the spectrum. FIG. 3C is a spectral distribution illustrating the relative emission intensity of UVC LEDs at wavelengths along the spectrum.

As shown in FIG. 3A, the action spectra for the SARS-CoV-2 virus includes a peak at 260 nm (corresponding to the peak absorbance of the nucleic acids of the genome) and a second peak or shoulder at wavelengths below 240 nm (where both nucleic acids and proteins absorb). As shown in FIG. 3B, LPM lamps emit almost monochromatic radiation at a characteristic wavelength of 253.7 nm. As shown in FIG. 3C, UVC LEDs emit radiation between 265 nm and 280 nm with the highest virucidal effectiveness occurring at 280 nm. Accordingly, in embodiments configured to deactivate the SARS-CoV-2 virus, the UVC radiation source 400 may be an LPM lamp or UVC LEDs.

In some embodiments, the UVC radiation source 400 may be a polychromatic radiation source (e.g., an array of LEDs or other UVC radiation sources 400 having a series of different wavelengths). In those embodiments, the UVC radiation source 400 may emit a broad spectrum of UV radiation (to deactivate a wide array of potential particles) or the controller 210 may output control signals to selectively emit UV radiation at a specific wavelength (to selectively deactivate a specific particle). In other embodiments, the protective respirator 100 may be configured to utilize any of a number of modular UVC radiation sources 400 having different wavelengths, enabling the protective respirator 100 to be configured and reconfigured to deactivate specific particles using each of the module UVC radiation sources 400.

FIG. 4A is a schematic diagram of a reactor chamber cavity 420 according to an exemplary embodiment wherein the UVC radiation source 400 is an LPM lamp 400a. FIG. 4B is a schematic diagram of a reactor chamber cavity 420 according to another exemplary embodiment wherein the UVC radiation source 400 is another LPM lamp 400b. FIG. 4C is a diagram of a reactor chamber cavity 420 according to another exemplary embodiment wherein the UVC radiation source 400 is an array of UVC LEDs 400c.

As shown in FIGS. 4A and 4B, the LPM lamp 400a or 400b may be located within the reactor chamber cavity 420 to emit UVC radiation within the reactor chamber cavity 420. As shown in FIG. 4C, the UVC LEDs 400c may be mounted on the outside of the reactor chamber cavity 420 and emit UVC radiation through holes in the reactor chamber cavity 420. As also shown in FIG. 4C, the reactor chamber cavity 420 may include baffles 410 to increase the time it takes for particles to traverse the length of the reactor chamber cavity 420 and maximize deactivation of potentially harmful particles.

LPM lamps 400a and 400b have several advantages, most notably lower thermal output and electrical efficiency that is multiple times higher than UV LEDs 400c. On the other hand, LPM lamps 400a and 400b require high voltages and do not have instantaneous start-up.

UV LEDs 400c have poor electrical efficiency (3-5%), require thermal management to mitigate the large heat profile, and are higher cost than LPM lamps 400a or 400b. However, UV LEDs 400c are rapidly becoming more efficient, have a higher power density (allowing arrays to be built so the system can scale easily, and are more durable than LPM lamps 400a or 400b (i.e., they are hermetically sealed and resistant to shock and vibration). Finally, because UV LEDs 400c are a directional UV radiation source 400, it is easier to control where the UV radiation is emitted.

As described in detail below, the protective respirator 100 is configured to prolong the battery life of the power source 290 while maintaining a threshold probability of deactivating at least one particle by maximizing germicidal and energy efficiency. To that end, the interior surface of the reactor chamber cavity 420 may consist of or may be coated with a UV reflective material 480. The reflective material 480 may be, for example, stainless steel, galvanized ducting, a specialized UV reflective aluminum, a microporous expanded polytetrafluoroethylene (ePTFE) material, etc. Specialized UV reflective aluminums can reflect up to 76 percent of UVC radiation. Meanwhile, microporous ePTFE materials can reflect up to 98 percent of UVC radiation.

Sumpner's Principle of Irradiance in a closed system states that the total the irradiance E within the reactor chamber cavity 420 is equal to the sum of the direct irradiance ED from the UVC radiation source 400 and the indirect irradiance ER that is reflected off the reflective material 480. The indirect irradiance ER is calculated by comparing the indirect irradiance ER to an ideal direct irradiance ED with no losses, defined as:

ER=ED×(R/(1−R))

where R is the reflectance of the reflective surface 480. Due to that relationship, a small increase in reflectance R of the reflective surface 480 translates into exponential increases in average total irradiance E.

In preferred embodiments, the reflective material 480 is a microporous sintered expanded PTFE (ePTFE), which has a near-perfect diffuse (Lambertian) reflectance and is resilient to UV degradation. Experiments with 254-nm UVC sources 400 and ePTFE reflective material 480 have recorded amplification of up to 20 times in the UV spectral range. Because of the exponential relationship between reflectivity and irradiance, increasing the reflectivity from 90 percent to 96 percent results in a larger gain than increasing the reflectance R from 70 percent to 90 percent. Accordingly, the increase of reflectivity between a stainless steel surface compared to reflective ePTFE material has been shown to increase irradiance by a factor approximately 10.

In some embodiments, the reflective surface 480 may be electrostatically treated to collect particles for extended UVC dosing and inactivating certain threats. For example, a nano coating of reagents may enhance the effectiveness of the reactor 12—with reactive photocatalysis.

As described above, each reactor chamber 120 includes an input port 121 for receiving untreated air 110 and an output port 129 for outputting treated air 190. At both of those ingress and egress locations, there is a potential loss of photons and reduced optical efficiency, which can contribute to a reduction of fluence inside the reactor chamber cavity 420. Accordingly, each reactor chamber cavity 420 may include two internal fluid-permeable photon barriers 500 (consisting of or coated with the reflective material 480) that reflect UV radiation back into the reactor chamber cavity 420, while allowing airflow to pass through the fluid-permeable photon barrier 500 with little obstruction.

FIG. 5A is a diagram illustrating fluid-permeable photon barriers 500 according to a first exemplary embodiment.

As shown in FIG. 5A, each fluid-permeable photon barrier 500 may include a series of venetian blind-like slats (or other baffles) that reflect UV photons back into the reactor chamber cavity 420 while allowing the intake air 110 or the exhaust air 190 to pass through the fluid-permeable photon barrier 500.

FIG. 5B is a front view of a fluid-permeable photon barrier 500 according to a second exemplary embodiment. FIG. 5C is a side view of the fluid-permeable photon barriers 500 according to the embodiment of FIG. 5B.

As shown in FIGS. 5B and 5C, each fluid-permeable photon barrier 500 may include a series of frustoconical shaped holes 520 that cause UVC radiation to propagate according to Knudsen molecular flow. The holes 520 may be designed to minimize UVC radiation propagation losses while also minimizing the gas-dynamic losses, which can reduce or eliminate the efficiency gains of using reflective materials 480. For instance, to ensure a low pressure drop across the fluid-permeable photon barrier, the total hole size may be greater than 40 percent of the cross-sectional area of the inlet aperture.

In some embodiments, the protective respirator 100 is configured to provide a threshold probability (e.g., 90 percent, 99 percent, 99.9 percent, etc.) of deactivating a specific particle (e.g., SARS-CoV-2, etc.) travelling through the reactor chamber cavity 420. The probability p of deactivating a specific particle (or the proportion of those particles deactivated) during irradiation depends on the fluence D of the UVC radiation and the susceptibility constant k of the specific particle:

p=1−e^{D*k}

Meanwhile, the fluence D of the UVC radiation depends on the intensity I of the UVC radiation along the path of that particle and over the time t that the particle is exposed to that UVC radiation:

D=∫I(t)·dt

To provide the threshold probability p of deactivating the particle, each reactor chamber cavity 420 may be configured to continuously emit UVC radiation at a constant intensity I. In preferred embodiments, however, the protective respirator 100 includes several features to prolong the battery life of the power source 290. Most notably, the protective respirator 100 may use approximately half the power by alternating between activating and deactivating the inhalation reactor chamber cavity 420a and the exhalation reactor chamber cavity 420b depending on whether the user 101 is inhaling or exhalating.

FIG. 6 is a flowchart illustrating a UV emission process 600 according to an exemplary embodiment.

As shown in FIG. 6, the controller 210 identifies whether the user 101 in inhaling or exhaling in step 610. In response to a determination that the user 101 is inhaling, the controller 210 enables the inhalation reactor chamber 120a in step 624 and disables the exhalation reactor chamber 120b in step 626. Alternatively, in response to a determination that the user 101 is exhaling, the controller 210 disables the inhalation reactor chamber 120a in step 634 and enables the exhalation reactor chamber 120b in step 636.

Additionally, the velocity of particles traveling through the reactor chamber cavity 420—and, by extension, the time t that those particles are exposed to UVC radiation—is dependent on the intensity of the respiration of the user 101. Therefore, in some embodiments, the controller 210 may estimate the intensity of the user's inhalation in step 640 and, in step 650, adjust the intensity I of the UV radiation output by the inhalation reactor chamber 120a or the exhalation reactor chamber 120b (for instance, to provide the threshold probability p of deactivating the particle over the time t that those particles are exposed to UVC radiation).

In the embodiments described above wherein the inhalation sensor 270a and the exhalation sensor 270b measure the flow rate across each check valve 260a and 260b, the controller 210 can determine the intensity of the user's respiration based on the flow rates measured by each of the inhalation sensor 270a and the exhalation sensor 270b. In other embodiments, however, the inhalation sensor 270a and the exhalation sensor 270b may simply output a signal indicating whether the check valve 260a or 260b is open or closed. Accordingly, in those embodiments, the controller 210 may estimate the intensity of the user's respiration based on the activity level of the user (e.g., based on data from one or more physiological sensors 240, the geolocation module 224, and/or the inertial measurement unit 250), the elevation of the user 101 (determined, for example, by the geolocation module 224), or information indicative of the user's physiological condition (e.g., sex, weight, height, smoking status, pulmonary well-being, etc.) stored, for example, in the memory 218.

The process 600 is recursive to repeatedly activate and deactivate each reactor chamber 120. The controller 210 may also monitor the temperature of the reactor chamber 120 in step 602 (and reduce the intensity I of the UVC radiation if the temperature of the reactor chamber 120 exceeds a predetermined threshold) and monitor the power supply 290 in step 604 (and reduce the intensity I of the UVC radiation if the remaining power is below a predetermined threshold).

FIG. 7A is a graph of an example respiration pattern 720. FIG. 7B is an example timing diagram of a binary signal 740 indicating the timing of the respiration pattern 740.

In the embodiments described above wherein the inhalation sensor 270a and the exhalation sensor 270b measure the flow rate across each check valve 260a and 260b, the controller 210 can identify the respiration pattern 720—including both the timing and the estimated intensity of respiration—based on the flow rates measured by each of the inhalation sensor 270a and the exhalation sensor 270b. In other embodiments, each check valve 260a and 260b the inhalation sensor 270a and the exhalation sensor 270b output a signal when the associated check valve 260a or 260b is opened or closed. In those embodiments, the signals output by the inhalation sensor 270a and the exhalation sensor 270b can form the binary signal 740. In other embodiments, the protective respirator may include a microphone and the controller may use signal processing algorithms (e.g., received stored in memory 218) to identify inhalations and exhalations based on sounds of the user breathing captured by the microphone. In still other embodiments, the controller 210 may identify each inhalation and exhalation by predicting those inhalations and exhalations based on the activity level of the user (e.g., based on data from one or more physiological sensors 240, the geolocation module 224, and/or the inertial measurement unit 250), the elevation of the user 101 (determined, for example, by the geolocation module 224), or information indicative of the user's physiological condition (e.g., sex, weight, height, smoking status, pulmonary well-being, etc.) stored, for example, in the memory 218. For example, the controller 210 may store a number of profiles (e.g., in memory 218) associated with a number factors (e.g., activity level of the user, elevation, physiological condition of the user) and associated with a signal similar to the binary signal 740 (e.g., a sine wave, a square wave, a saw tooth wave). In those instances, the controller 210 may predict the inhalations and exhalations of the user by selecting a stored profile and identifying the signal associated with that stored profile.

FIG. 7C is a timing diagram of pulses 760 to activate the inhalation reactor chamber 120a and pulses 780 to activate the exhalation reactor chamber 120b in response to the respiration pattern 720 of FIG. 7A or the binary signal 740 of FIG. 7B according to exemplary embodiments.

To determine the required intensity I of UVC radiation to achieve the predetermined probability p to deactivate the particle, the controller 210 may use a mathematical model (stored, for example, in the memory 218) developed based on laboratory experiments and computational fluid dynamics. The mathematical model may be any description of the system by a set of variables (e.g., real numbers, boolean values, etc.) and a set of relationships between those variables. The mathematical model may be, for example, a formula or a look-up table (stored, e.g., in memory 218) for determining the required intensity I of UVC radiation to achieve the predetermined probability p to deactivate the particle in view of the variables described herein (e.g., the intensity of the respiration of the user, etc.).

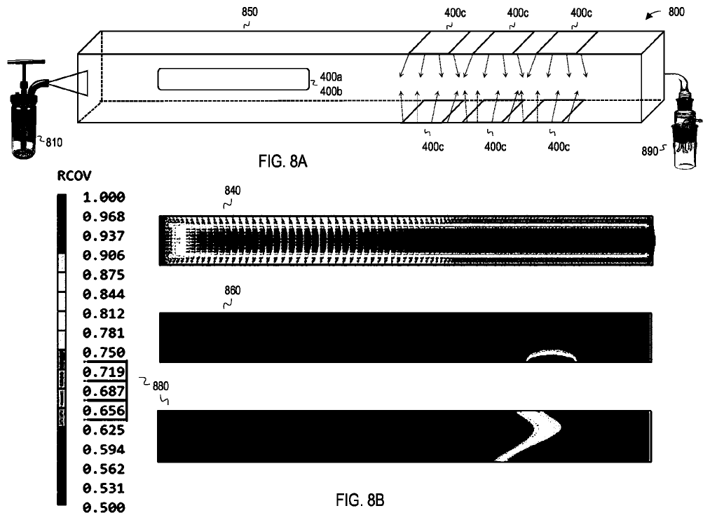

FIG. 8A illustrates an example laboratory experiment 800 to measure the intrinsic kinetics of the inactivation response of aerosolized particles.

In the example laboratory experiment 800, aerosolized particles are introduced into a quartz channel 850 using a nebulizer 810. The quartz channel 850 includes a UVC radiation source 400 (e.g., an LPM lamp 400a or 400b or UVC LEDs 400c). The aerosolized particles are exposed to the UVC radiation as they pass through the quartz channel 850. Samples are collected at the output of the quartz channel 850 using a bioaerosol sampler 890. Those samples are then analyzed to determine the inactivation response of the aerosolized particles that are exposed to the UVC irradiation.

To determine the inactivation response of known viruses, the laboratory experiments are conducted using biological surrogates for those pathogens. For example, experiments are conducted using phages that have the ability to infect bacteria but no ability to affect human tissues. To determine the inactivation response of coronaviruses, for instance, phages are selected that have responses to UV radiation that are similar to those coronaviruses. For instance, the T1 and T1UV phage, the Φ6 phage, the Qβ phage, and the mouse hepatitis virus (MHV) all have similar log-linear (first order) behavior and are all slightly conservative for coronaviruses.

Having measured the intrinsic kinetics of the inactivation responses of known pathogens using those laboratory experiments, the inactivation responses of those known pathogens are then simulated by a computer model using computational fluid dynamics.

FIG. 8B illustrates a computer simulation of the inactivation response during the experiment 800. Heat map 840 is a map of the velocity vector field through the quartz channel 850. Image 860 is a map of the fluence rate contours of one of the UVC radiation source 400. Integrating that velocity field and the fluence rate of the UVC radiation source 400 with the kinetics of inactivation for a specific pathogen enables the system to develop a mathematical model predicting the inactivation response of the specific pathogen, as shown in image 880, as it passes through the channel 850.

In embodiments where the UVC radiation source 400 is a LPM lamp 400a or 400b, each reactor chamber 120 may include a ballast to preheat and ignite the LPM lamp 400a or 400b and activate or deactivate the LPM lamp 400a or 400b (or adjust the intensity I of the UVC radiation) by dimming the LPM lamp 400a or 400b.

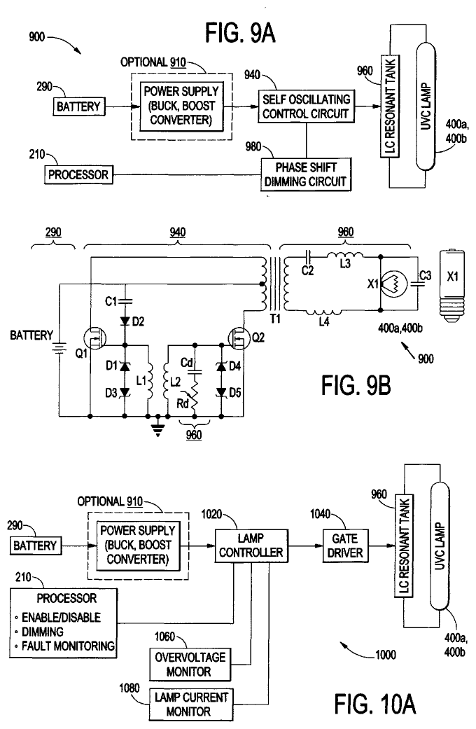

FIG. 9A is a block diagram of an analog ballast 900 for a LPM lamp 400a or 400b according to an exemplary embodiment. FIG. 9B is a circuit diagram of the analog ballast 900 of FIG. 9A.

As shown in FIGS. 9A and 9B, the ballast 900 includes an LC resonant tank 960, a self-oscillating control circuit 940, and a dimming circuit 980. As shown in FIGS. 9A and 9B, the ballast may include a power boosting circuit 910 to boost the power supplied by the power supply 290. The LC resonant tank 960 oscillates the LPM lamp 400a or 400b at the resonant frequency of the LPM lamp 400a or 400b. The LC resonant tank 960 also provides feedback across the transformer T1 to turn on and off the transistors Q1 and Q2 of the self-oscillating control circuit 940, which in turn drives the LC resonant tank 960. The dimming circuit 980 includes a variable resistor Rd and the capacitor Cd. The controller 210 controls the intensity of the UVC radiation by adjusting the resistance of the variable resistor Rd. The addition of the capacitor and the resistor network reduces the slope of the magnetizing current and the current through the RC network, resulting in a negative correlation to operating frequency. Therefore, a smaller resistor Rd will result in the higher the lamp power. As the lamp frequency moves above resonance, that reduces the Q-factor and reduces current, resulting in a dimming effect.

Resonant LC circuits are ideal due to the non-linear behavior of CCFL and MPL lamps 400a and 400b, providing an inexpensive, reliable, and efficient control structure.

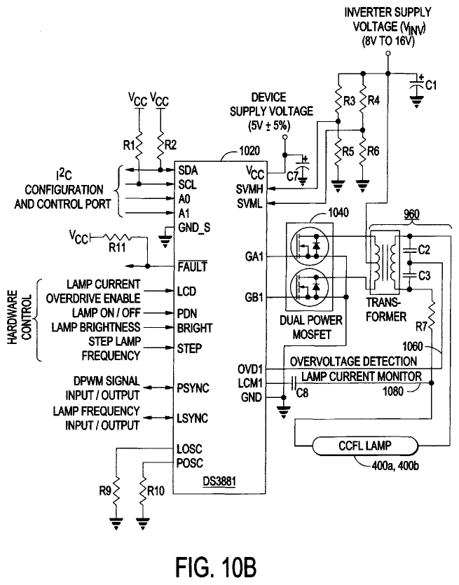

FIG. 10A is a block diagram of a ballast 1000 for a LPM lamp 400a or 400b having an integrated circuit-based lamp controller 1020 according to an exemplary embodiment. FIG. 10B is a circuit diagram of the IC-based ballast 1000 of FIG. 10A.

A push-pull drive scheme generates a low voltage DC signal to a high voltage AC signal required to drive a low pressure mercury lamp. By adjusting the MOSFET on-time, the current is regulated and additional burst dimming is achieved by using a digital pulse-width modulated signal. The IC-based ballast 1000 does not rely on resonant frequency; instead, the lamp controller 1020 sets the lamp frequency and current feedback for frequency adjustment.

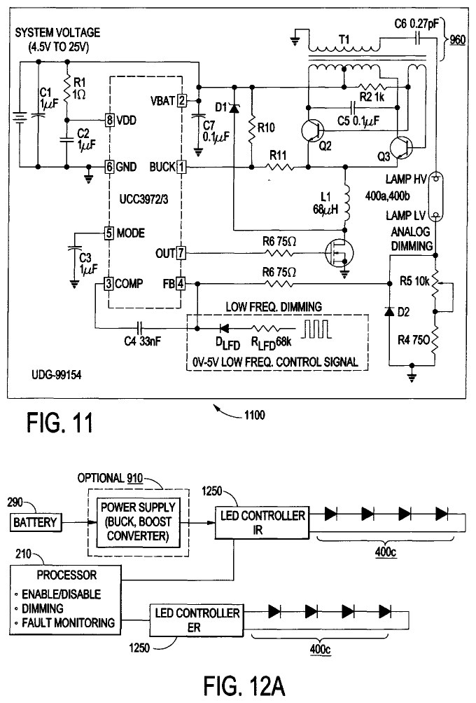

FIG. 11 is a block diagram of a ballast 1100 for an LPM lamp 400a or 400b according to another exemplary embodiment. A current-fed push-pull topology is used to power the LPM mercury lamp 400a or 400b. The circuit consists of a buck stage, a resonant push-pull stage, and an output stage. The buck power stage includes a Metal Oxide Field Effect Transistor Sbuck, flyback diode Dbuck, and an inductor Lbuck. The integrated ballast controller 1020 matches the buck frequency with the push-pull stage. The LC resonant tank 960 produces a sinusoidal voltage and causes the output of the buck stage to become a full-wave rectified voltage referenced to Vbat. The transistors Q1 and Q2 are alternatively driven by a resonant frequency and floating AC voltage with an auxiliary winding on the transformer T1. The transistors Q1 and Q2 operate at a 50% duty cycle and supply current from the buck inductor into each side of the LC resonant tank 960 at the resonant frequency. The LPM lamp 400a or 400b is driven through the LC resonant tank 960 with sinusoidal currents and voltages and is driven from a DC current source Ibuck. The Transformer T1 amplifies the sinusoidal tank voltage to generate a sinusoidal secondary voltage that is divided between the LPM lamp 400a or 400b and the ballast capacitor.

The IC-based ballasts 1000 and 1100 are more complex and higher cost than the analog ballast 900 but offer improved dimming control by adjusting the phase angle or by using an integrated buck converter to vary the lamp voltage directly. The IC-based lamp controllers 1020 also have additional housekeeping and safety features such as overvoltage, zero voltage detection, open lamp detection, soft-start, etc. Some lamp controllers 1020 even have feedback to the controller 210 to control the chip or monitor faults.

In embodiments where the UVC radiation source 400 is UVC LEDs 400c, the intensity I of the UVC radiation may be adjusted by adjusting the current through the UVC LEDs 400c. In preferred embodiments, however, the intensity I of the UVC radiation may be adjusted by pulsing the UVC LEDs 400c and modulating the pulse width to adjust the ratio of time when the UVC LEDs 400c emit UVC radiation relative to the time when the UVC LEDs 400c do not emit UVC radiation.

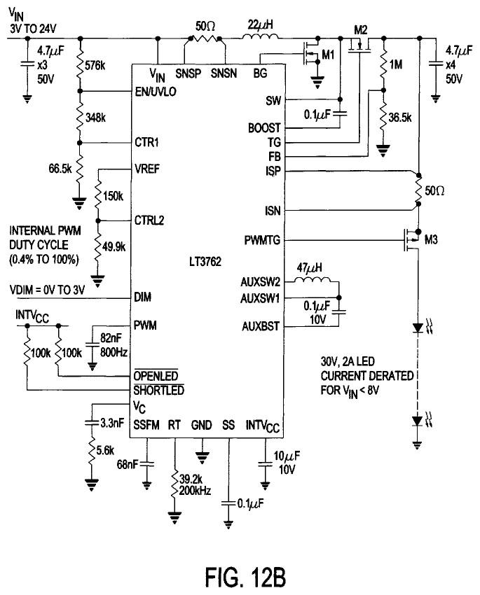

FIG. 12A is a block diagram illustrating circuit controllers 1250 adjusting the intensity of UVC LEDs 400c according to another exemplary embodiment. FIG. 12B is a circuit diagram illustrating the circuit controllers 1250 of FIG. 12A.

The LT3762 features a synchronous DC/DC boost converter and constant current source output chip. It provides superior efficiency for driving high powered LEDs 400c with low losses from a battery source 290, making it suitable for the protective respirator without additional components and a dedicated power supply. The controller 210 provides two signals to the LED driver 1250, an enable signal to turn on the LEDs 400c and a PWM generated dimming signal to the chip which ratiometrically adjusts the LED forward current.

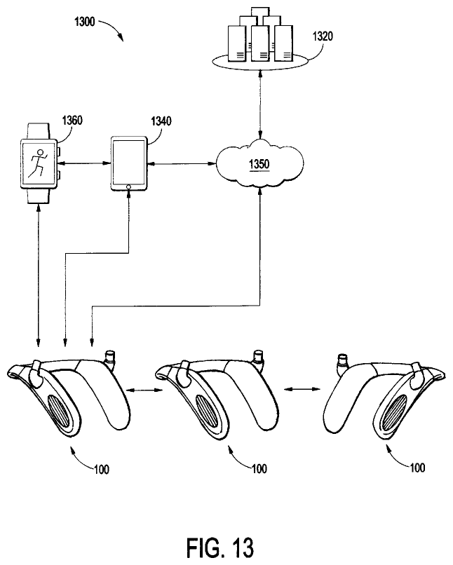

FIG. 13 is a diagram of a network environment 1300 according to exemplary embodiments.

As shown in FIG. 13, the protective respirator 100 may communicate with a server 1320 (e.g., using the communications module 220) over one or more wide area networks 1050 such as the internet. In some embodiments, each protective respirator 100 is configured to communicate with other protective respirators 100 (e.g., via Bluetooth, a local area network, a cell network, etc.) to form a mesh network. Additionally or alternatively, the protective respirator 100 may pair with a personal electronic device 1340 (e.g., a smartphone) using a direct, wireless communications protocol (e.g., Bluetooth). In those embodiments, the protective respirator 100 may communicate with the server 1320 via the personal electronic device 1340 and/or a mesh network of personal respirators 100. In some of those embodiments, the personal respirator 100 and/or the personal electronic device 1340 may also be paired with an activity tracker 1360 (e.g., a fitness tracker, wristband, smartwatch, etc.). Meanwhile, in those embodiments, the geolocation module 224, the physiological sensors 240, and/or the IMU 250 described above with reference to FIG. 2 may be incorporated in the personal electronic device 1340 and/or activity tracker 1360.

By communicating with the server 1320, the protective respirator 100 is able to receive the mathematical model (developed using computational fluid dynamics and the intrinsic kinetics of inactivation identified in laboratory experiments as described above) used by the controller 210 to determine the required intensity I of UV-C radiation to deliver the required fluence D to achieve a predetermined probability p of deactivating a specific particle (e.g., SARS-CoV-2) as it passes through each reactor chamber cavity 420. The protective respirator 100 may also upload performance data, usage data, and other telemetric data to the server 1320 for further aggregation and analysis.

In some of those embodiments, multiple protective respirators 100 in close proximity (for example, soldiers or airmen in the same vehicle or transport plane) may work collectively to achieve the predetermined probability p of deactivating the specified particle in the surrounding atmosphere. In those embodiments, the protective respirators 100 may determine the number of protective respirators 100 in close proximity (e.g., using short-range direct wireless messages, infrared or other proximity detection, etc.) and determine the required intensity I of UVC radiation—as dictated by the mathematical model described above—for the protective respirators 100 to collectively achieve the predetermined probability p of deactivating the specified particle.

In other embodiments, the protective respirator 100 may determine the required intensity I of UVC radiation to achieve the predetermined probability p of deactivating the specified particle based on the number of people in close proximity (determined, for example, using infrared or other proximity detection, etc.).

In some embodiments, the required intensity I of UVC radiation for the protective respirator(s) 100 to achieve the predetermined probability p of deactivating the specified particle may be based on an estimated concentration of the specified particle in the geographic location of the protective respirator 100. For instance, the controller 210 may receive an indication that a specified geographic area may include a relatively high concentration of the specified particle, determine (e.g., using the geolocation module 224) that the protective respirator is within the specified geographic area, and adjust the intensity I of UVC radiation as described above to predetermined probability p of deactivating the specified particle in untreated intake air 110a with the relatively high concentration of the specified particle.

In the embodiments described above, the protective respirator 100 is a “symmetrical flow” disinfection device that includes both an inhalation reactor chamber 120a and an exhalation reactor chamber 120b. In some embodiments, the protective respirator 100 may have a low power mode wherein only the inhalation reactor chamber 120a is activated (for example, in situations in which the power supply 290 is low and/or the user 101 is alone). In still other embodiments, the protective respirator 100 may include only the inhalation reactor chamber 120a for providing treated intake air 190a to the user.

While preferred embodiments have been described above, those skilled in the art who have reviewed the present disclosure will readily appreciate that other embodiments can be realized within the scope of the invention. Accordingly, the present invention should be construed as limited only by any appended claims.

What is claimed is:

1. A symmetrical flow respirator, comprising:
an inhalation reactor chamber, comprising:
a first reactor chamber cavity that receives untreated intake air from an atmosphere surrounding a user; and
a first ultraviolet radiation source that irradiates the untreated intake air to form treated intake air for inhalation by the user; and
an exhalation reactor chamber, comprising:
a second reactor chamber cavity that receives untreated exhaust air exhaled by the user; and
a second ultraviolet radiation source that irradiates the untreated exhaust air to form treated exhaust air for dissipation into the atmosphere surrounding the user.

2. The respirator of claim 1, further comprising a controller configured to:
identify an inhalation of the user;
output a control signal causing the inhalation reactor chamber to irradiate the untreated intake air in response to the inhalation of the user;
identify an exhalation of the user; and
output a control signal causing the exhalation reactor chamber to irradiate the untreated exhaust air in response to the exhalation of the user.

3. The respirator of claim 1, wherein the controller is further configured to:
output a control signal deactivating the exhalation reactor chamber in response to the inhalation of the user; and
output a control signal deactivating the inhalation reactor chamber in response to the exhalation of the user.

4. The respirator of claim 2, wherein the controller is further configured to:
identify an intensity of the inhalation of the user; and
output a control signal adjusting an intensity of the ultraviolet radiation that irradiates the untreated intake air in accordance with the intensity of the inhalation of the user.

5. The respirator of claim 4, wherein the controller uses a mathematical model to select the intensity of the ultraviolet radiation based on the intensity of the inhalation of the user.

6. The respirator of claim 5, wherein the controller:
identifies the inhalation of the user in response to a signal output by an inhalation sensor that detects an opening of a check valve of the inhalation reactor chamber; and
identifies the exhalation of the user in response to a signal output by an exhalation sensor that detects an opening of a check valve of the exhalation reactor chamber.

7. The respirator of claim 6, wherein:
the inhalation sensor is an inhalation flow rate sensor that detects the intensity of the inhalation of the user by detecting a flow rate of the treated intake air.

8. The respirator of claim 2, wherein the controller is configured to identify the inhalation of the user and the exhalation of the user by predicting the inhalation of the user and the exhalation of the user based on an activity level of the user, the elevation of the respirator, or information indicative of the physiological condition of the user.

9. The respirator of claim 1, wherein interior surfaces of the first reactor chamber cavity and the second reactor chamber cavity comprise a reflective material for reflecting ultraviolet radiation.

10. The respirator of claim 1, wherein each of the first reactor chamber cavity and the second reactor chamber cavity comprise two fluid-permeable photon barriers that reflect ultraviolet radiation while allowing airflow to pass through the fluid-permeable photon barrier.

11. A method of deactivating at least one particle, by a symmetrical flow respirator, the method comprising:
receiving untreated intake air from an atmosphere surrounding a user by a first reactor chamber cavity;
irradiating the untreated intake air, by a first ultraviolet radiation source, to form treated intake air;
outputting the treated intake air for inhalation by the user;
receiving untreated exhaust air, exhaled by the user, by a second reactor chamber cavity; and
irradiating the untreated exhaust air by a second ultraviolet radiation source.

12. The method of claim 11, further comprising:
identifying an inhalation of the user; and
identifying an exhalation of the user,
wherein the untreated intake air is irradiated in response to the inhalation of the user and the untreated exhaust air is irradiated in response to the exhalation of the user.

13. The method of claim 11, further comprising:
deactivating the exhalation reactor chamber in response to the inhalation of the user; and
deactivating the inhalation reactor chamber in response to the exhalation of the user.

14. The method of claim 12, further comprising:
identifying an intensity of the inhalation of the user; and
adjusting an intensity of the ultraviolet radiation that irradiates the untreated intake air in accordance with the intensity of the inhalation of the user.

15. The method of claim 14, selecting the intensity of the ultraviolet radiation based on the intensity of the inhalation of the user using a mathematical model.

16. The method of claim 15, further comprising:
detecting an opening of a check valve of the inhalation reactor chamber; and
detecting an opening of a check valve of the exhalation reactor chamber,
wherein the inhalation of the user is identified based on the opening of the check valve of the inhalation reactor chamber and wherein the exhalation of the user is identified based on the opening of the check valve of the exhalation reactor chamber.

17. The method of claim 16, wherein the intensity of the inhalation of the user is identified by detecting a flow rate through the check valve of the inhalation reactor chamber.

18. The method of claim 12, wherein identifying the inhalation of the user and the exhalation of the user comprises predicting the inhalation of the user and the exhalation of the user based on an activity level of the user, the elevation of the respirator, or information indicative of the physiological condition of the user.

19. The method of claim 11, wherein interior surfaces of the first reactor chamber cavity and the second reactor chamber cavity comprise a reflective material for reflecting ultraviolet radiation.

20. The method of claim 11, wherein each of the first reactor chamber cavity and the second reactor chamber cavity comprise two fluid-permeable photon barriers that reflect ultraviolet radiation while allowing airflow to pass through the fluid-permeable photon barrier.
